(19) United States
(12) Patent Application Publication
(10) Pub. No.: US 2019/0362828 A1
(43) Pub. Date: Nov. 28, 2019

(54) SYSTEMS AND METHODS FOR ELECTRONIC PRESCRIPTIONS

(71) Applicant: Kevin Laxer, Los Angeles, CA (US)

(72) Inventor: Kevin Laxer, Los Angeles, CA (US)

(21) Appl. No.: 15/987,725

(22) Filed: May 23, 2018

(51) Int. Cl. G16H 20/10; G16H 10/60; G06Q 10/08; G06Q 30/06; H04L 29/06; G01S 19/51; G01S 1/68

(52) U.S. Cl. CPC G16H 20/10; G16H 10/60; G06Q 10/087; G01S 1/68; G06Q 30/0611; H04L 63/083; G01S 19/51; G06Q 30/0609

(57) ABSTRACT

The invention refers to a system for a portable electronic prescription includes receiving login credentials of a user for an electronic prescription database, the electronic prescription database is a decentralized storage network, wherein the decentralized storage network includes a digital ledger, validating the user's credentials with the electronic prescription database, receiving a prescription for the user, using the prescription in a search of the electronic prescription database to obtain a list of pharmacies with the lowest prices for the prescription and the location of the listed pharmacies, sending the listed pharmacies, their prices and locations for display to the user, receiving a pharmacy intent based on a selection by the user, and completing a transaction between the user and the selected pharmacy.

### BACKGROUND OF THE INVENTION

### Field of the Invention

Embodiments of the invention relates to systems and methods for electronic prescriptions to improve the traditional prescription process.

### Description of the Related Art

There are currently no known systems that provide electronic prescription in such a way to simplify the prescription process by allowing the user to transport their prescription with them. Current systems for prescriptions entail going to the doctor's office and receiving a prescription.

The user does not know whether that pharmacy has the prescription available and at what cost. This is particularly problematic in places, where the user is on a limited budget. In such cases, the user is at the mercy of the pharmacy to offer a competitive price for the prescription. In addition, the user does not know whether the pharmacy even has the prescription on stock to sell to the public.

This is particularly problematic because many times, it is the sick, poor, and elderly who needs the drug. Moreover, it would be burdensome for the user to go from on pharmacy to another to look for the availability of their prescription. Without the ability to find the lowest prices, a truly competitive system cannot be enacted.

In addition, there may be issues of security and the fear of hacking or corruption. The issue of security needs to be balanced with a system with a real-time updated ledger that is able to track the latest patient or user information.

To overcome the problems and limitations described above there is a need for a system provides, in real-time, a competitive price for the prescription and inventory of the prescription. Moreover, there is a need for a system which allows the pharmacies to bid on the prescriptions of individuals while still maintain profitability for their company and their shareholders. As such, a system that is able to compile a list of pharmacies that have the desired prescription, an inventory of the prescription, all the while using a bidding system employed by the pharmacies to offer the lowest price is needed. Moreover, a system where a real-time ledger that is secure and is able to track the changes that are made to an electronic prescription database is needed.

### BRIEF SUMMARY OF THE INVENTION

One or more embodiments of the invention are directed a systems and methods for electronic prescriptions.

In one or more embodiments, a method for a portable electronic prescription includes receiving login credentials of a user for an electronic prescription database, the electronic prescription database is a decentralized storage network, wherein the decentralized storage network includes a digital ledger, validating the user's credentials with the electronic prescription database, receiving a prescription for the user, using the prescription in a search of the electronic prescription database to obtain a list of pharmacies with the lowest prices for the prescription and the location of the listed pharmacies, sending the listed pharmacies, their prices and locations for display to the user, receiving a pharmacy intent based on a selection by the user, and completing a transaction between the user and the selected pharmacy.

The prescription may include an electronically converted handwritten prescription.

The prescription may include the user's medical records.

The user's prescription may be refillable.

The electronic prescription database may include a real-time inventory of the prescription.

The listed pharmacies may further include the user's last used pharmacy.

The location of the listed pharmacy may be determinable by global positioning satellite.

The location of the listed pharmacy may be determinable by geofencing.

The login credentials may include the user's Username and Password for the electronic prescription database.

The prescription may include one or more of a first name, last name, date of birth, social security number, driver's license number, domicile, medication prescription, and prescription history.

In one or more embodiments, a method for a portable electronic prescription includes receiving login credentials of a user for an electronic prescription database, the electronic prescription database is a decentralized storage network, wherein the decentralized storage network includes a digital ledger, validating the user's credentials with the electronic prescription database, receiving a prescription for the user, locating a pharmacy proximate to a location of the user, using the prescription in a search of the electronic prescription database to obtain a list of pharmacies with the lowest prices for the prescription, real-time internal inventory, the location of the listed pharmacies, and driving directions to the listed pharmacies, sending the listed pharmacies, their prices, real-time internal inventories, locations, driving directions for display to the user, receiving the user's pharmacy intent based on a selection by the user, and completing the transaction between the user and the selected pharmacy.

The portable electronic prescription may include an electronically converted handwritten prescription.

The prescription may include the user's medical records.

The user's portable electronic prescription may be refillable.

The listed pharmacies may include the user's last used pharmacy.

The location of the listed pharmacy is determinable by global positioning satellite.

The location of the listed pharmacy may be determinable by geofencing.

The login credentials may include the user's Username and Password for the electronic prescription database.

The prescription may include one or more of a first name, last name, date of birth, social security number, driver's license number, domicile, medication prescription, and prescription history.

In one or more embodiments, a method for bidding for a prescription includes receiving login credentials of a pharmacy for an electronic prescription database, the electronic prescription database is a decentralized storage network, wherein the decentralized storage network includes a digital ledger, validating the pharmacy's credentials with the electronic prescription database, sending the prescription for a user, using a location of the user to locate a proximate pharmacy, checking a real-time internal inventory of the pharmacy proximate to the location of the user, bidding with a price for the prescription based on a predetermined pricing matrix with one or more drug manufacturers, receiving a bid with the price of the prescription and real-time internal inventory level of the prescription for the pharmacy proximate to the location of the user for display to the user, sending a user's pharmacy intent based on a selection by the user, and completing the transaction between the user and the selected pharmacy.

### BRIEF DESCRIPTION OF THE DRAWINGS

The above and other aspects, features and advantages of the invention will be more apparent from the following more particular description thereof, presented in conjunction with the following drawings wherein:

FIG. 1 is a flowchart illustrating the process of verifying and receiving information about pharmacies that that have the prescription in stock and the price in accordance with one or more embodiments of the present invention.

FIG. 2 is a flowchart illustrating the process of verifying and receiving information about pharmacies that that have the prescription in stock and the price in accordance with one or more embodiments of the present invention.

FIG. 3 is a flowchart illustrating the process of bidding and sending information about prescriptions in stock and the price in accordance with one or more embodiments of the present invention.

FIG. 4 illustrates a general-purpose computer and peripherals that when programmed as described herein may operate as a specially programmed computer capable of implementing one or more methods, apparatus and/or systems of the present invention.

FIG. 5 is an exemplary sign up graphical user interface for log into the system in accordance with one or more embodiments of the present invention.

FIG. 6 is a flowchart illustrating the process of acquiring a subject's information in accordance with one or more embodiments of the present invention.

FIG. 7 is an exemplary graphical user interface for selection of identification method of the subject in accordance with one or more embodiments of the present invention.

FIG. 8A is an exemplary graphical user interface for using a subject's driver's license for identification in accordance with one or more embodiments of the present invention.

FIG. 8B is an exemplary graphical user interface for using the barcode on a subject's prescription for identification in accordance with one or more embodiments of the present invention.

FIG. 8C is an exemplary graphical user interface for using text fields to enter a subject's identification in accordance with one or more embodiments of the present invention.

FIG. 8D is an exemplary graphical user interface for using a subject's paper prescription to be scanned into the system in accordance with one or more embodiments of the present invention.

FIG. 9A is an exemplary graphical user interface for displaying the pharmacy inventory in accordance with one or more embodiments of the present invention.

FIG. 9B is an exemplary graphical user interface for displaying the user's pharmacy history in accordance with one or more embodiments of the present invention.

FIG. 9C is an exemplary graphical user interface for displaying the user's pharmacy history in accordance with one or more embodiments of the present invention.

FIG. 10 is an exemplary graphical user interface for displaying options for the user to choose in accordance with one or more embodiments of the present invention.

FIG. 11 is an exemplary graphical user interface showing history of past prescriptions in accordance with one or more embodiments of the present invention.

FIG. 12 is an exemplary entered insurance info graphical user interface for the systems in accordance with one or more embodiments of the present invention.

FIG. 13A is an exemplary graphical user interface for using text fields to enter a user's personal information in medical history in accordance with one or more embodiments of the present invention.

FIG. 13B is an exemplary graphical user interface displaying a user's medical history in accordance with one or more embodiments of the present invention.

FIG. 14 is an exemplary graphical user interface showing a selection of proximate pharmacies in accordance with one or more embodiments of the present invention.

FIG. 15 is an exemplary graphical user interface showing a prescription overview in accordance with one or more embodiments of the present invention.

FIG. 16 is an exemplary graphical user interface confirming payment information in accordance with one or more embodiments of the present invention.

FIG. 17 is an exemplary graphical user interface verifying the selection of the pharmacy and the prescription in accordance with one or more embodiments of the present invention.

FIG. 18 is an exemplary graphical user interface confirming the selection of the pharmacy and the prescription in accordance with one or more embodiments of the present invention.

FIG. 19 is an exemplary graphical user interface reminding the user to pick up the prescription in accordance with one or more embodiments of the present invention.

FIG. 20 is an exemplary graphical user interface verifying that the prescription is picked up in accordance with one or more embodiments of the present invention.

FIG. 21 is an exemplary graphical user interface displaying direction directions to the pharmacy in accordance with one or more embodiments of the present invention.

FIG. 22 is an exemplary graphical user interface displaying direction directions to the pharmacy while using geofencing in accordance with one or more embodiments of the present invention.

FIG. 23 illustrates conceptually the blockchain aspect of the electronic prescription database as described herein capable of implementing one or more methods, apparatus and/or systems of the present invention.

### DETAILED DESCRIPTION

The present invention comprising systems and methods for electronic prescriptions will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. Furthermore, although steps or processes are set forth in an exemplary order to provide an understanding of one or more systems and methods, the exemplary order is not meant to be limiting. One of ordinary skill in the art would recognize that the steps or processes may be performed in a different order, and that one or more steps or processes may be performed simultaneously or in multiple process flows without departing from the spirit or the scope of the invention. In other instances, specific features, quantities, or measurements well known to those of ordinary skill in the art have not been described in detail so as not to obscure the invention. It should be noted that although examples of the invention are set forth herein, the claims, and the full scope of any equivalents, are what define the metes and bounds of the invention.

For a better understanding of the disclosed embodiment, its operating advantages, and the specified object attained by its uses, reference should be made to the accompanying drawings and descriptive matter in which there are illustrated exemplary disclosed embodiments. The disclosed embodiments are not intended to be limited to the specific forms set forth herein. It is understood that various omissions and substitutions of equivalents are contemplated as circumstances may suggest or render expedient, but these are intended to cover the application or implementation.

The term “first”, “second” and the like, herein do not denote any order, quantity or importance, but rather are used to distinguish one element from another, and the terms “a” and “an” herein do not denote a limitation of quantity, but rather denote the presence of at least one of the referenced item.

For the purposes of this application the term pharmacy may be used to describe an independent pharmacy, a large pharmaceutical corporation, or a chain of pharmacies. The term user may be used to describe a patient, a patient's guardian, or anyone else for whom the prescription was written and was empowered to act on that person's behalf.

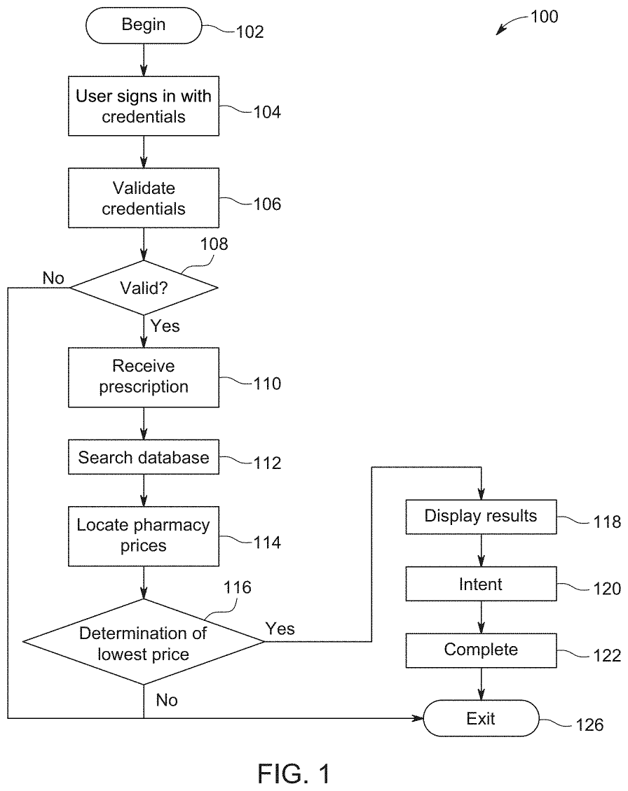

One or more embodiments of the present invention will now be described with references to FIGS. 1-23.

FIG. 1 is an illustration of a method 100 for a portable electronic prescription with one or more embodiments of the present invention. As illustrated, the process begins at step 102 by a user launching a software application (“App”) for the system of the present invention on a smart device, e.g. a smartphone, tablet, laptop, etc. A smart device with wireless, e.g., cellular or Wi-Fi, capability is preferable because the user may connect to the required database in real-time to have up to date history of the subject in relation to prescriptions.

In step 104, the App launches a user interface for the user to log into the system with their prescription database credentials, e.g. identification and password, fingerprint, facial recognition, etc. The user may be a patient, an individual, or group/organization that has a prescription from a physician or any other prescription source.

In the following descriptions, a portable electronic prescription is to be used for illustrative purposes only. Those of skill in the art would appreciate that the systems and methods described herein would be applicable to other types of industries and subject matters and thus are not limited to embodiments described herein.

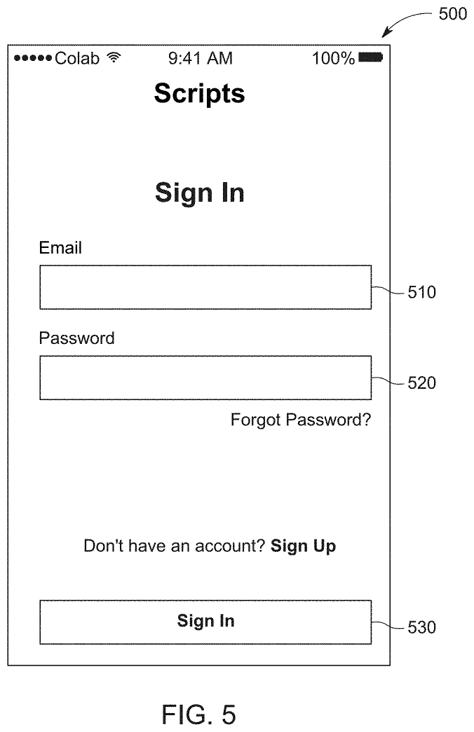

FIG. 5 is an exemplary login graphical user interface 500 for a user to log into the system in accordance with one or more embodiments of the present invention. As illustrated, the user enters their email address 510 or some other type of identification. In the illustrated example, the user enters their password to log into the database. In other embodiments, the user may log in with their name, social security number, nickname, or fingerprint or facial scan or any other identifying method.

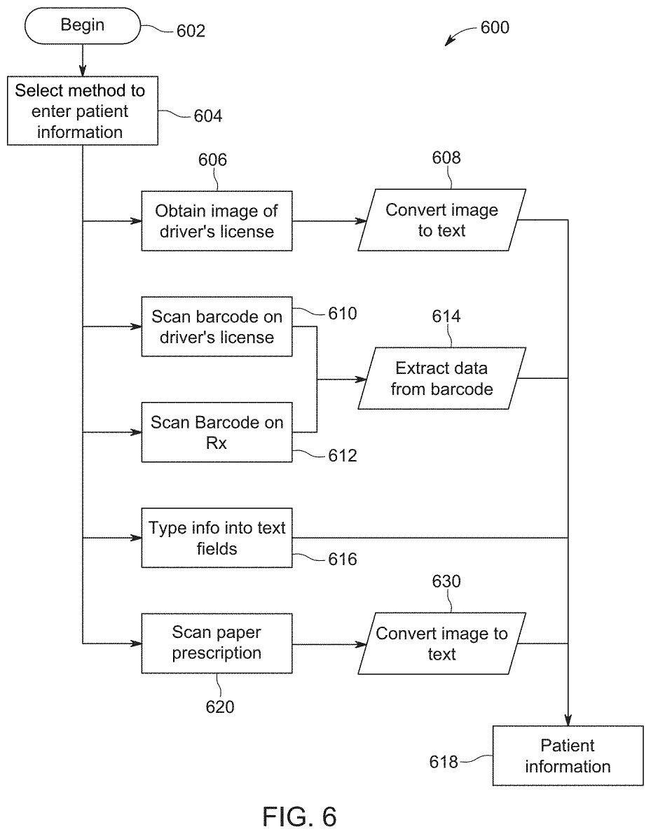

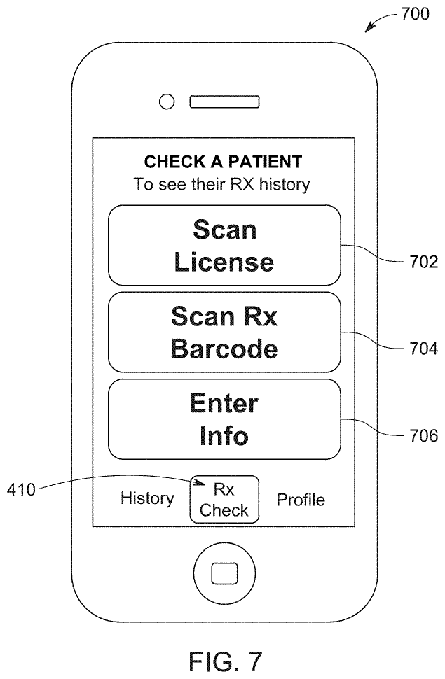

FIG. 6 is a flowchart illustrating the process 600 of acquiring a subject's information in accordance with one or more embodiments of the present invention. As illustrated, the process begins at step 602, and at step 604 the user is prompted to select a method to enter the subject's identification. FIG. 7 is an exemplary graphical user interface (GUI) 700 for the user to select an identification method for the subject in accordance with one or more embodiments of the present invention. In this illustrated example, the user may select button 702 to scan the subject's Driver's License, button 704 to scan a prescription (Rx) barcode, or button 706 to manually enter the required information.

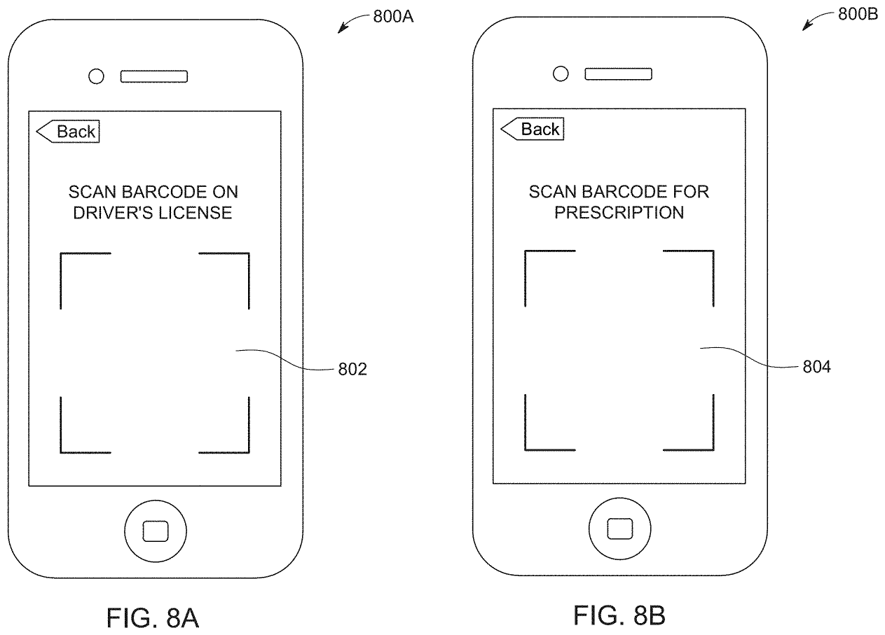

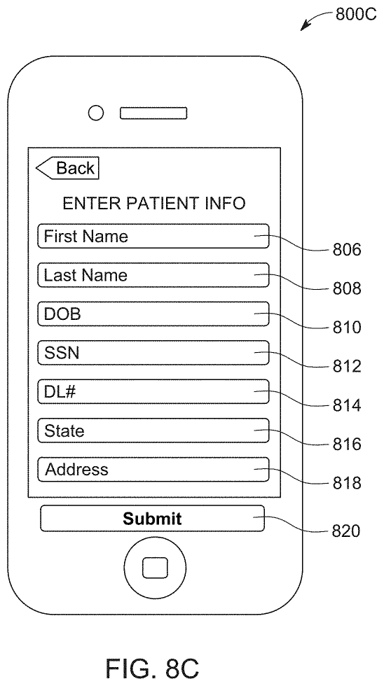

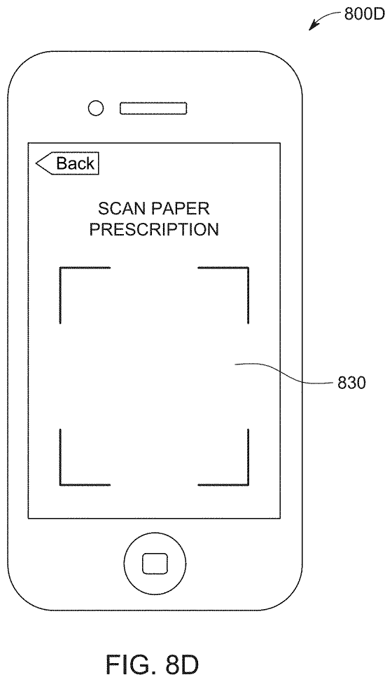

If the user selects button 702, for example, the subject's identification information may be acquired from his/her state's driver's license, e.g., by image recognition in block 606 and converting the image to text in block 608. Image recognition may be performed by capturing an image of the subject's driver's license using, for example, the graphical user interface 800A, illustrated in FIG. 8A. The subject's image may be captured using a camera on a smartphone, for example. An alternate method of acquiring the subject's identification information may by reading the barcode on the subject's driver's license image 800A in block 610. The subject's identification data 618 is subsequently extracted from the barcode in block 614.

Returning back to FIG. 7, if the user selects button 704, for example, the subject's identification information may be acquired from his/her prescription in block 612. An image of the subject's prescription may be captured, for example, using the graphical user interface 800B, illustrated in FIG. 8B. The subject's identification data 618 is subsequently extracted from the barcode in block 614.

If in GUI 700, as illustrated in FIG. 7, the user selects button 706, for example, the subject's identification information may be acquired by the user manually entering the required information into text fields in step 616. FIG. 8C is an exemplary graphical user interface 800C for using text fields to enter a subject's identification in accordance with one or more embodiments of the present invention. As illustrated, the user may enter information into fields 806 for first name, 808 for last name, 810 for date of birth, 812 for social security number, 814 for driver's license number, and the subject's domicile, e.g., 816 for the subject's state of resident, and 820 for the subject's address. After entering at least one of the data 806-818 as the subject identification data 618, the user selects the Submit button 820.

Returning back to FIG. 1, in step 106, the system validates the user's login information, i.e., credentials, with an email address 510 and password 520, after selection of the Sign In button 530. In step 108, if a determination is made that the user is not a valid user, then the system may exit at step 126 or return to step 104 for the user to reenter the correct login information.

However, if at step 108 a determination is made that the user is a valid user, the system proceeds to step 110 to obtain the user's prescription.

After the user's credentials are validated, using any of the above methods, or any other available method, the system proceeds to step 110 to receive the prescription. The prescription may be entered in any number of ways. As a non-limiting example, the prescription may be a standard paper prescription prescribed by the doctor's office.

The standard paper prescription may be scanned and turned into an electronic prescription. FIG. 8D is an exemplary graphical user interface 800D for a user to scan a standard paper prescription in accordance with one or more embodiments of the present invention. The standard paper prescription information may be converted from his/her prescription, e.g., by image recognition in block 620 and converting the image to text in block 630. Image recognition may be performed by capturing an image of the user's prescription using, for example, the graphical user interface 800D, illustrated in FIG. 8D. The prescription may be captured by using a camera on a smartphone, for example. An alternate method of acquiring the user's prescription information may be accomplished by reading a barcode on the user's prescription image 800B in block 612. The user's prescription data 612 is subsequently extracted from the barcode in block 614.

In other embodiments, the prescription may be an electronic prescription issued by the physician's office. The electronic prescription may contain the user's medical history or records, insurance information, the user's past prescription that may interaction with other drugs, and including how many times the prescription has been fulfilled.

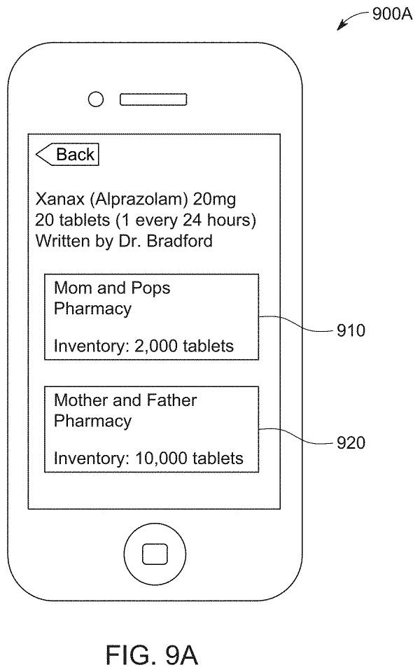

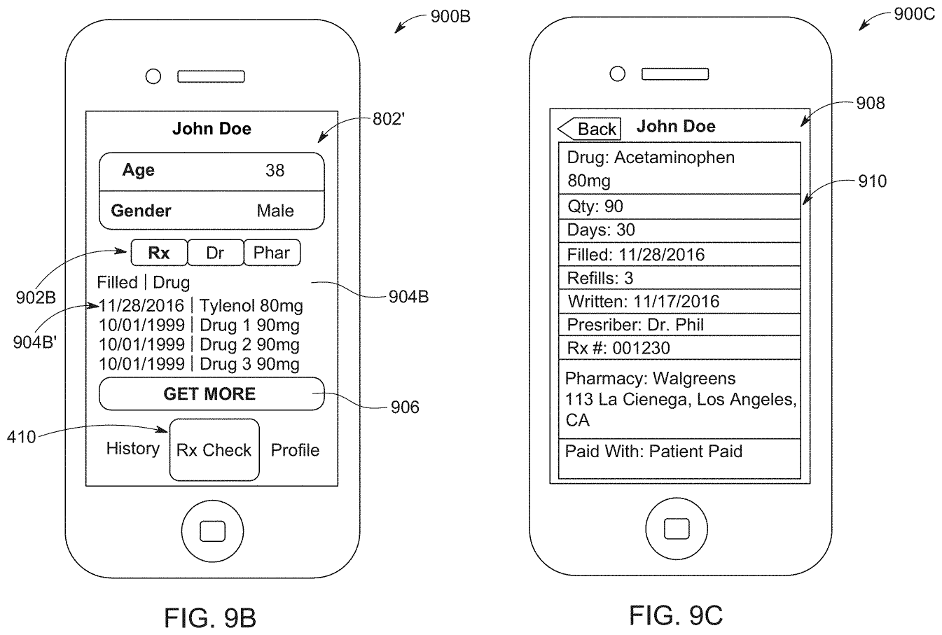

FIG. 9A is an exemplary display graphical user interface 900A for the user to see the inventory 910 in accordance with one or more embodiments of the present invention.

FIG. 9B is an exemplary graphical user interface 900B showing prescription history for the subject in accordance with one or more embodiments of the present invention. As illustrated, the subject's identification information is displayed in field 802′. Field 902B provides for selection of the type of information the user desires to be displayed in window 904B. For instance, the user may review prior prescriptions by selecting “Rx”; the doctors that provided the prior prescriptions by selecting “Dr”; or the pharmacy that filled the prescription by selecting “Phar”. The “GET MORE” button 906 provides for scrolling to additional hidden information that did not fit in window 904B.

In one or more embodiments, the user may review any of the listed items in window 904B by selecting it, e.g. 904B′. Upon selection, details of the selected prescription intent are presented as exemplified in FIG. 9C, which is an exemplary graphical user interface 900C showing details of a selected prior prescription intent of the user in accordance with one or more embodiments of the present invention. As illustrated, GUI 900C includes field 908 for the subject's identification; and window 910 for the details of the prescription intent. Window 910 may include one or more of the following fields: the name of the “Drug”; the quantity (“Qty”); the number of days for the prescription; the date the intent was “Filled”; the number of “Refills” available; the prescribing doctor (“Prescriber”); the prescription number (“Rx #”); the pharmacy that filled the prescription; and how it was paid for.

Thus, the search in the database in step 112 may return a history of unfilled prescriptions for the user, which is up-to-date.

Once the prescription is received, one skilled in the art would appreciate that the system's database may be a secured database that is preferably governed by and meets all regulatory requirements.

In one or more embodiments, the database may contain a listing of available prices for the prescription 114. Thus, the search in the database in step 112 may return a list of real-time inventories of the desired prescription that may be proximate or near the location of the user.

Moreover, the step in 114 may return a list of the prices of the received prescription. As such, the database may return a list of the pharmacies that are near the location of the user along with the lowest prices of the prescription. As a non-limiting example, the list may include the top three pharmacies that are nearest to the user, has the lowest prices, and where the prescription is currently available in the local inventory. In another embodiment, the list may be adjusted by the user and may result in more than three pharmacies and extend the area of the geographic region.

In step 116, when a determination is made as to the availability of the prescription and the pharmacies that are closest to the user, the system may exit at step 126 or proceed to step 118 for the system to display the results.

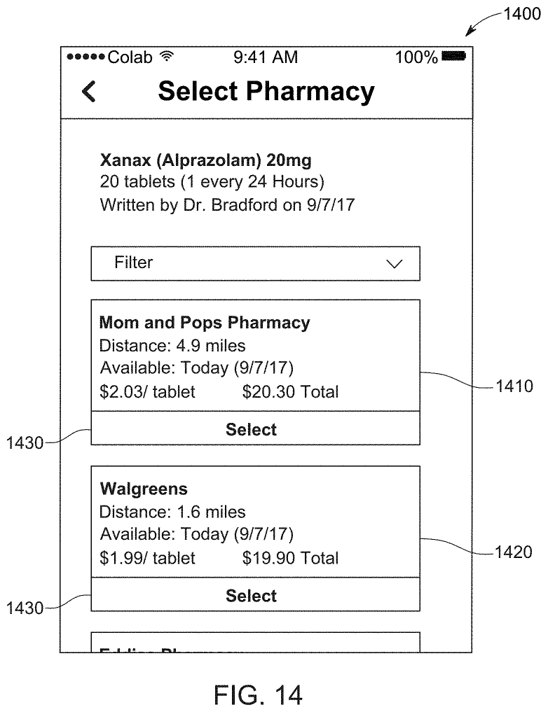

FIG. 14 is an exemplary display graphical user interface 1400 in accordance with one or more embodiments of the present invention. As a non-limiting example, the results may provide the user with a first option 1410, a second option 1420, or any other number of options.

Returning back to FIG. 1, if at step 116 a determination is made of the available pharmacies with the particular prescription with the lowest prices in the database, the system may proceed to step 118 to display a list of the aforementioned pharmacies to the user and to request prescriptions intent. As illustrated in FIG. 14, once the user makes a decision as to which pharmacy to use, the user may press the “select” button 1430 or any other button that may be used to signify the user's choice or intent 120.

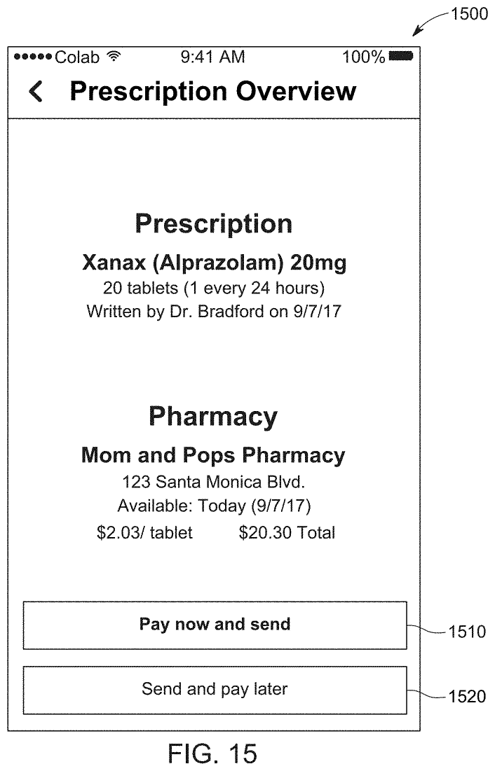

Returning back to FIG. 1, once the desired pharmacy is chosen or selected, the system proceeds to step 122 to complete the transaction and may proceed to collect payment, as illustrated in FIG. 15.

FIG. 15 is a non-limiting example of a graphical user interface 1500 for the user to select a payment method for the subject in accordance with one or more embodiments of the present invention. In this illustrated example, the user may select to pay now and send the prescription 1510 or the user may send the prescription and pay later 1520.

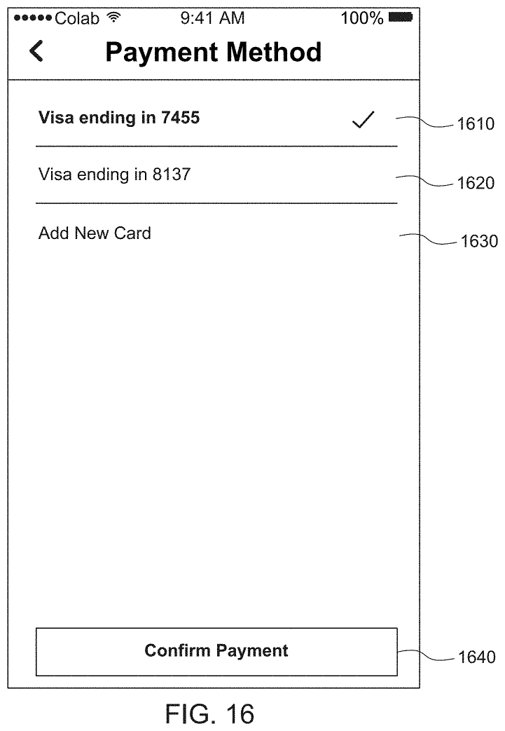

If the user selects to pay now and send 1510, for example, the user may see an example of a graphical user interface 1600 shown in FIG. 16. The user may have already entered his or her payment information previously. In the current example, the user may have already entered a credit card number 1610 or entered another credit card number 1620. The user may select one of these options to reuse that particular card. In another example, the user may enter another card, one that has not previously been entered. As such, the user may select the add new card 1630 and enter the new card's information. As a non-limiting example, the user may manually type in the requested credit card information, e.g., name, account number, expiration date, etc. In another embodiment, the user may take a scan of the card and thereby auto-populate the information. Regardless of which card is chosen, the user may press the confirm payment button 1640 to confirm the payment information.

Returning back to FIG. 15, if the user selects to send and pay later 1520, then the prescription intent is sent to the selected pharmacy. The selected pharmacy may receive the pharmacy intent and then prepare the prescription to be picked up. Once the user arrives at the pharmacy, the user may then pay for the prescription. As non-limiting examples, the user may pay with cash, credit card, or any other types of currency or items of value.

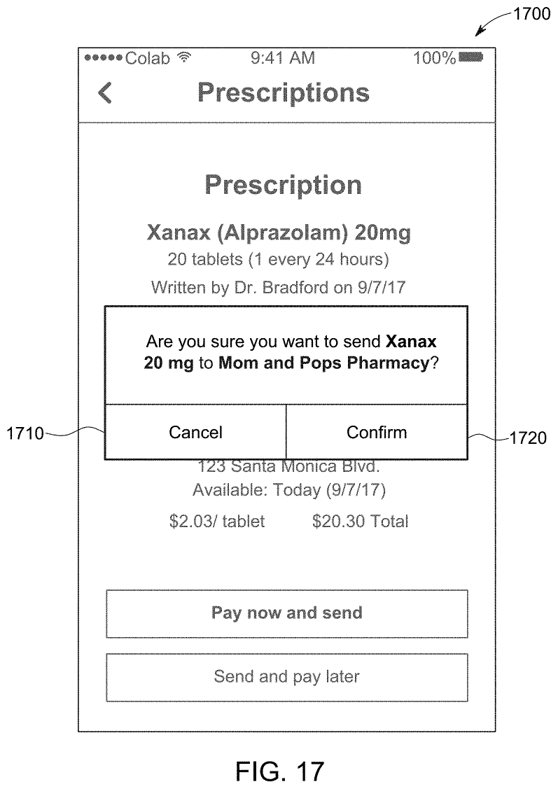

FIG. 17 is a non-limiting example of a graphical user interface 1700 for the user to confirm sending the prescription to the selected pharmacy in accordance with one or more embodiments of the present invention. In this illustrated example, the user may select to cancel the transaction 1710 or the user may send the prescription to the pharmacy 1720.

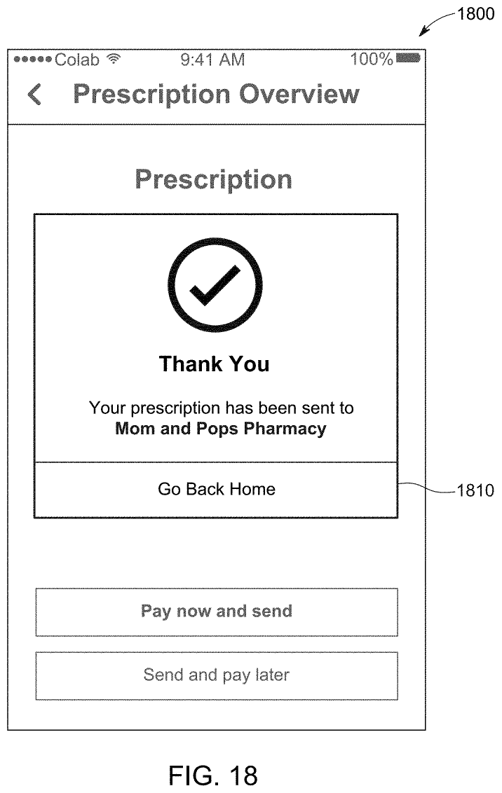

FIG. 18 is a non-limiting example of a graphical user interface 1800 thanking the user's selection and to confirm sending the prescription to the selected pharmacy in accordance with one or more embodiments of the present invention. In this illustrated example, the user may select to go back to the starting point or home 1810.

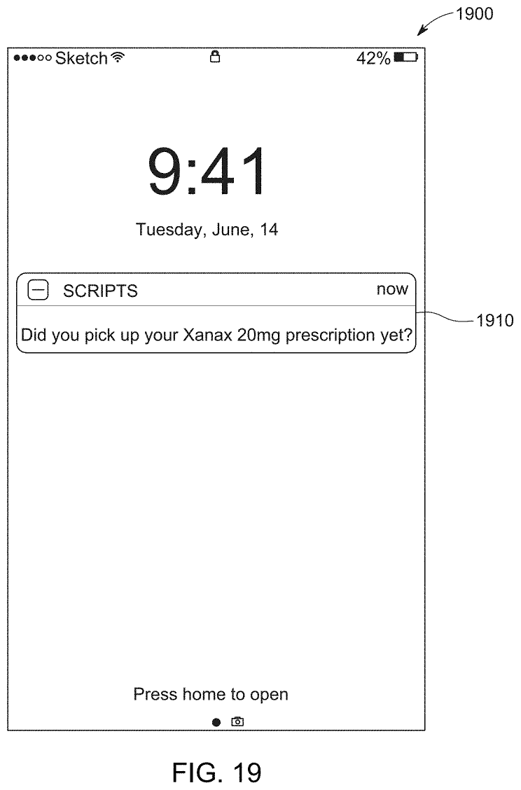

FIG. 19 is a non-limiting example of a graphical user interface 1900 asking the user if he or she has picked up their prescription in accordance with one or more embodiments of the present invention. This prompt may also act as a reminder in case the user has forgotten to pick up his or her prescription.

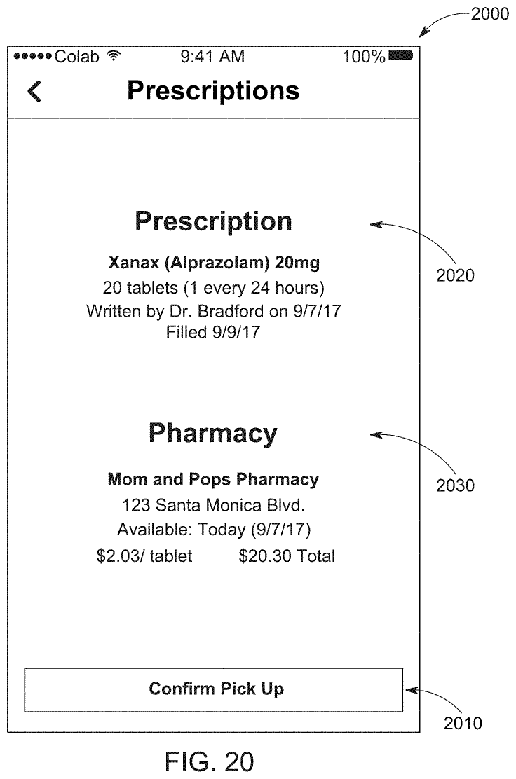

FIG. 20 is a non-limiting example of a graphical user interface 2000 if the user has responded to the prompt in FIG. 19 in accordance with one or more embodiments of the present invention. Returning back to FIG. 20, in this illustrated example, the reminder displays a summary of the prescription 2020 and the selected pharmacy 2030. When the user picks up the prescription, he or she may submit the confirm pick up button 2010 to prevent any further reminders.

Returning back to FIG. 1, once the transaction is chosen or selected, the system proceeds to step 126 to exit the system.

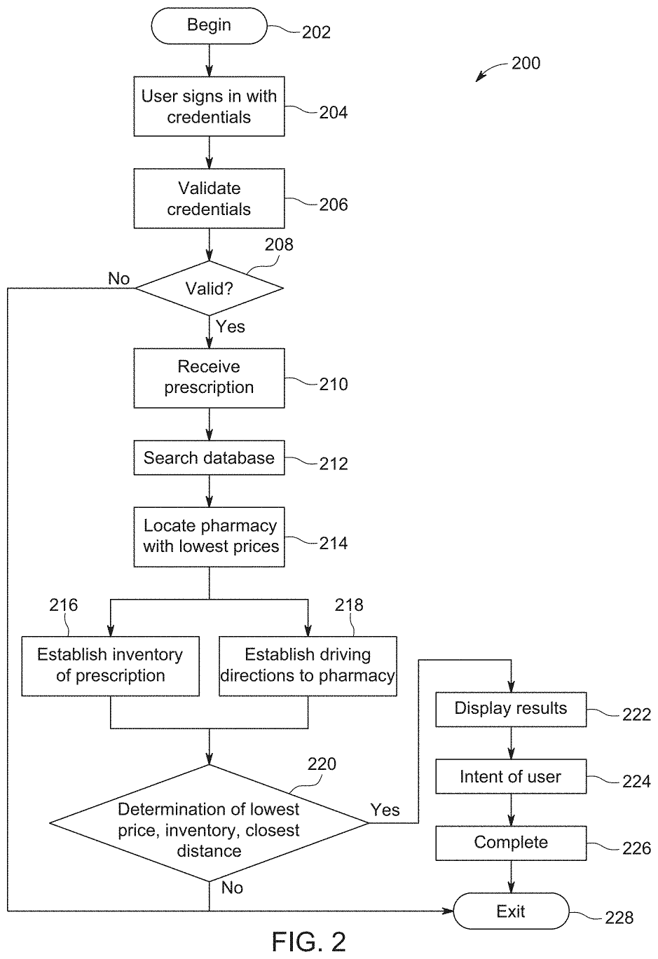

FIG. 2 is an illustration of a method 200 for a portable electronic prescription with one or more embodiments of the present invention. As illustrated, the process is similar to an embodiment as described in FIG. 1. However, in the present embodiment, the system is able to track the real-time inventory of each of the pharmacies. Most pharmacies have an accurate inventory of each drug or medication and this inventory may be placed in a company database. By using this system, access to such an inventory may be possible. Once the system has access to the database, the system would be aware of the real-time inventory of the particular drug or medication.

As illustrated, the process begins at step 202 by a user launching a software application (“App”) for the system of the present invention on a smart device, e.g. a smartphone, tablet, laptop, etc. A smart device with wireless, e.g., cellular or Wi-Fi, capability is preferable because the user may connect to the required database in real-time to have up to date history of the subject in relation to prescriptions.

In step 204, the App launches a user interface for the user to log into the system with their prescription database credentials, e.g. identification and password, fingerprint, facial recognition, etc. The user may be a patient, an individual, or group/organization that has a prescription from a physician or any other prescription source.

In the following descriptions, a portable electronic prescription is to be used for illustrative purposes only. Those of skill in the art would appreciate that the systems and methods described herein would be applicable to other types of industries and subject matters and thus are not limited to embodiments described herein.

In step 206, the system validates the user's login information. In step 208, if a determination is made that the user is not a valid user, then the system may exit at step 228 or return to step 204 for the user to reenter the correct login information.

However, if at step 208 a determination is made that the user is a valid user, the system proceeds to step 210 to obtain the user's prescription.

After the user's credentials are validated, using any of the above methods, or any other available method, the system proceeds to step 210 to receive the prescription. The prescription may be entered in any number of ways. As a non-limiting example, the prescription may be a standard paper prescription prescribed by the doctor's office.

Thus, the search in the database in step 212 may return a history of unfilled prescriptions for the user, which is up-to-date.

Once the prescription is received, one skilled in the art would appreciate that the system's database may be a secured database that is preferably governed by and meets all regulatory requirements.

In one or more embodiments, the database may contain a listing of available prices for the prescription 214. Thus, the search in the database in step 212 may return a list of real-time inventories of the desired prescription that may be proximate or near the location of the user.

Moreover, the step in 214 may return a list of the prices of the received prescription. As such, the database may return a list of the pharmacies that are near the location of the user along with the lowest prices of the prescription. As a non-limiting example, the list may include the top three pharmacies that are nearest to the user, has the lowest prices, and where the prescription is currently available in the local inventory. In another embodiment, the list may be adjusted by the user and may result in more than three pharmacies and extend the area of the geographic region.

FIG. 9 A is a non-limiting example of a graphical user interface 900 A informing the user of the real-time inventory of the drug or medication in accordance with one or more embodiments of the present invention. In this illustrated example, the user may display a certain pharmacy and display the inventory of the desired medication at that pharmacy 910. Another pharmacy with the desired medication may display the current inventory level 920.

In addition, in step 216, the system may establish an inventory of the medication in the pharmacy located in the database. In step 218, the system may establish driving directions to the listed pharmacies.

In step 220, when a determination is made as to the availability of the prescription, the inventory, lowest prices, and the pharmacies that are closest to the user, the system may exit at step 228 or proceed to step 22 for the system to display the results.

If at step 220 a determination is made of the available pharmacies with the particular prescription with the lowest prices, availability in the inventory, and the pharmacies that are closest to the user in the database, the system may proceed to step 222 to display a list of the aforementioned pharmacies to the user and to request prescriptions intent.

Once the desired pharmacy is chosen or selected 224, the system proceeds to step 226 to complete the transaction and may proceed to collect payment.

Returning back to FIG. 2, once the desired pharmacy is chosen or selected, the system proceeds to display driving directions to the pharmacy. As non-limiting examples, the system may work in conjunction with a global positioning system (GPS) to direct the user to the location of the pharmacy. As such, the GPS system may triangulate on the user's position by using their mobile device. In other embodiments, the user may use a system that directs the user to the pharmacy by any other means such as geofencing, log in information, etc.

If the system is able to locate the user by geofencing, the mobile device may trigger a response in the system to ascertain the location of the user. As such, if the user triggers a response by the system, the system would know the location of the user and find a pharmacy closest to the user.

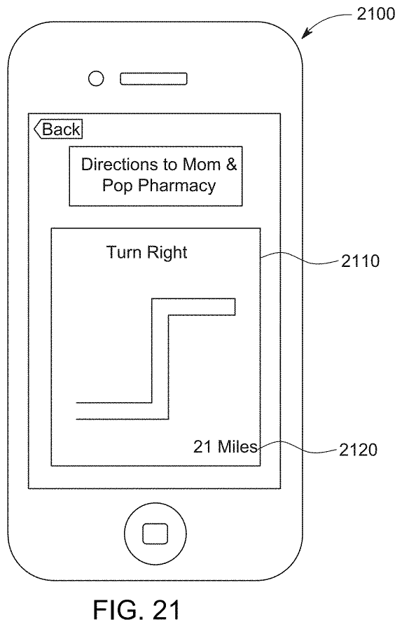

FIG. 21 is a non-limiting example of a graphical user interface 2100 giving the user direction to the chosen pharmacy in accordance with one or more embodiments of the present invention. In this illustrated example, the user may receive directions to the selected pharmacy 2110. Another feature of the driving directions may include the distance to the pharmacy 2120. The system may use geofencing to create a virtual geographic boundary, enabling the system to

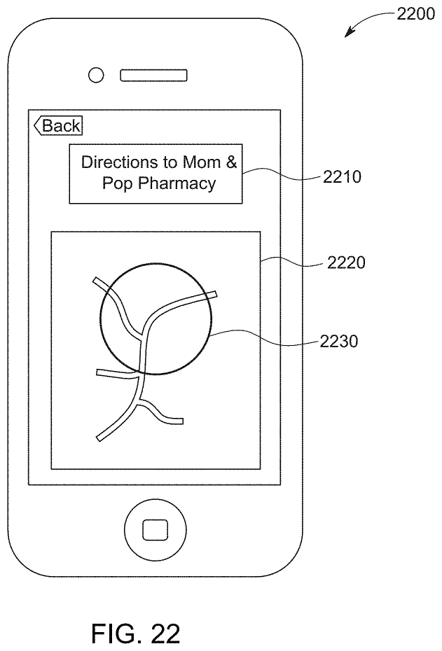

FIG. 22 is a non-limiting example of a graphical user interface 2200 giving the user direction to the chosen pharmacy in accordance with one or more embodiments of the present invention. In this illustrated example, the user may receive directions to the selected pharmacy 2210 by using geofencing. The system may use geofencing to create a virtual geographic boundary 2230, enabling the system to trigger a response when the system enters or leaves a particular area 2220.

Once the transaction is chosen or selected, the system proceeds to step 228 to exit the system.

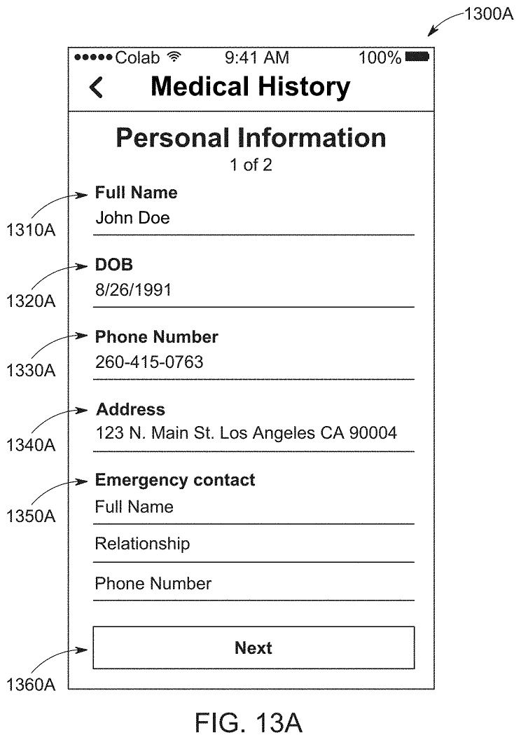

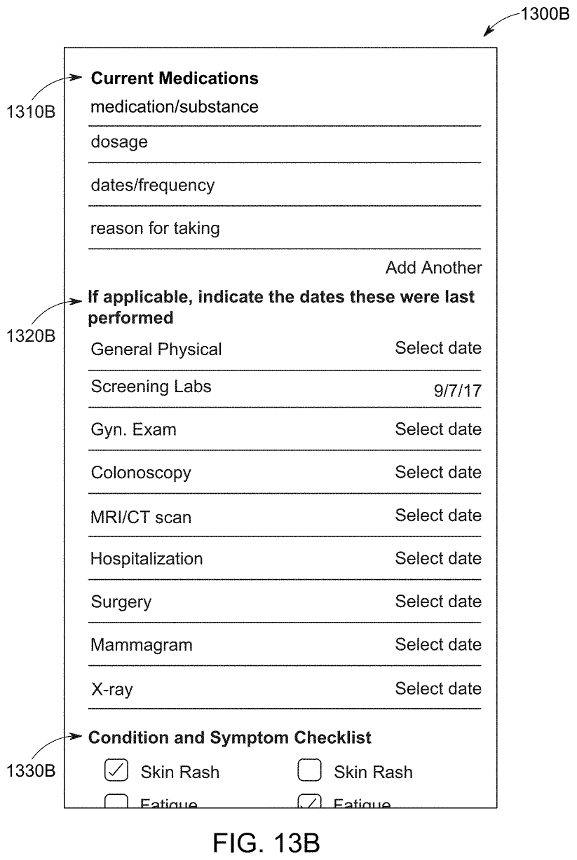

The system may have other features such as portable medical records. FIGS. 13 A and B are non-limiting examples of a graphical user interface 1300 A and B allowing the user to take their medical records with them in accordance with one or more embodiments of the present invention. As a non-limiting example, the medical records may contain the user's name, address, birthday, age, known allergies, report of the user's physical condition, blood pressure, resting heart rate, previous blood work test results, other test results, x-rays, MRI reports, CAT Scans, and any other medically or non-medically related information that pertains to the user. FIGS. 13 A and B will be reviewed more closely later in the present application.

In one or more embodiments, the system periodically reconciles the database with the one or more pharmaceutical databases. The reconciliation process minimizes duplication of data between the pharmaceutical databases and the database. Also, reconciliation serves the function of updating prescription intents in the local database that ultimately became filled prescriptions.

The previous descriptions were related to embodiments of the present invention from the perspective of the user. The following description is related to embodiments of the present invention from the perspective of the pharmacy. As such, pharmacies may bid on the prescriptions when the user enters a request.

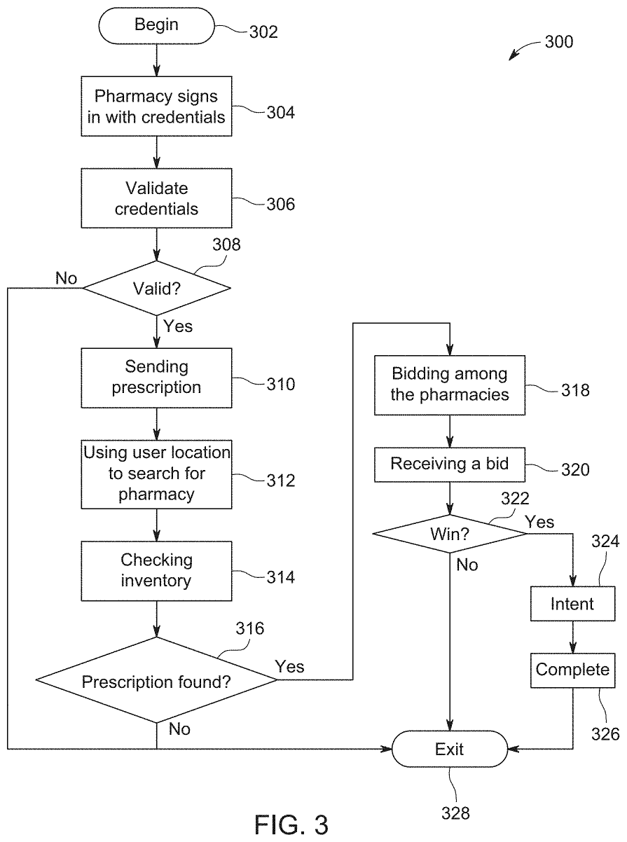

FIG. 3 is an illustration of a method 300 for a portable electronic prescription bidding feature by pharmacies with one or more embodiments of the present invention. As illustrated, the process begins at step 302 by a pharmacy launching a software application (“App”) for the system of the present invention on a smart device, e.g. a smartphone, tablet, laptop, etc. A smart device with wireless, e.g., cellular or Wi-Fi, capability may be preferable because the pharmacy may connect to the required database in real-time to have up to date history of the subject in relation to prescriptions.

In step 304, the App launches a user interface for the pharmacy to log into the system with their pharmacy database credentials, e.g., identification and password, fingerprint, facial recognition, etc. The pharmacy may be an independent pharmacy, a large pharmaceutical corporation, a chain of pharmacies or group/organization that is able to take prescriptions from a physician or any other prescription source.

In the following descriptions, a bidding system for prescriptions is to be used for illustrative purposes only. Those of skill in the art would appreciate that the systems and methods described herein would be applicable to other types of industries and subject matters and thus are not limited to embodiments described herein.

In one or more embodiments of the present invention a pharmacy may log into the system. The pharmacy enters their email address or some other type of identification. The pharmacy enters their password to log into the database. In other embodiments, the pharmacy may log in with their company name, company number, or any other identifying method. As a non-limiting example, the pharmacy may log in with some sort of security identification system.

In step 306, the system validates the pharmacy's login information, e.g., credentials, with an email address and password. In step 308, if a determination is made that the pharmacy is not a valid user, then the system may exit at step 328 or return to step 304 for the pharmacy to reenter the correct login information.

However, if at step 308 a determination is made that the pharmacy is a valid user, the system proceeds to step 310 to bid upon the prescription.

After the pharmacy's credentials are validated, using any of the above methods, or any other available method, the system proceeds to step 310 to receive the prescription request.

The standard paper prescription may be scanned and turned into an electronic prescription. In other embodiments, the prescription may be an electronic prescription issued by the physician's office. The electronic prescription may also contain the user's medical history or records. In yet another example, the electronic prescription may also contain the user's insurance information. Moreover, the electronic prescription may also contain the user's pharmacological history including how many times the prescription has been filled and the price point at which the prescription was sold.

In one or more embodiments, the database may contain a history of filled prescriptions and contain a history of prescription intents, e.g., unfilled prescriptions. Thus, the search in the database may return a history of unfilled prescriptions for the user, which is up-to-date. As such, if the number of filled prescriptions have exceeded the prescribed number, then the system may inform the pharmacy not to fulfill the prescription. In other non-limiting examples, the system may prevent the pharmacy from accidentally overriding the exceeded prescribed number of prescriptions by not even presenting an option to fulfill the prescription. However, for the purposes of this application, the description of an embodiment of the system will continue to discuss the subsequent steps after the system receives the prescription.

Once the prescription is received, one skilled in the art would appreciate that the system's database may be a secured database that is preferably governed by and meets all regulatory requirements.

It may not be feasible for the pharmacy to wait for prescription bids to come in, as such there may be an alternative. The pharmacy may have a preset system of prices for the prescription. As such, a matrix may be put in place where the pharmacy automatically bids for the prescription. As a non-limiting example, the prescription may have an initial bid of $50. However, when another pharmacy places a bid that is lower, the pharmacy may have the preset matrix where it takes into consideration, the availability of the drug, location, supply, and other factors and may place a lower bid in order to beat the $50 bid. In one or more embodiments, the database may contain a listing of available prices from the other pharmacies bidding for the prescription 314. Thus, the search in the database in step 312 may return a list of real-time inventories of the desired prescription that may be proximate or near the location of the user.

Moreover, the step in 314 may return a list of the prices of the received prescription. As such, the database may return a list of the pharmacies that are near the location of the user along with the lowest prices of the prescription. As a non-limiting example, the list may include the top three pharmacies that are nearest to the user, has the lowest prices, and where the prescription is currently available in the local inventory. In another embodiment, the list may be adjusted by the user and may result in more than three pharmacies and extend the area of the geographic region.

In step 316, when a determination is made as to the availability of the prescription and the pharmacies that are closest to the user, the system may exit at step 328 or proceed to step 318 for the pharmacies to place a bid. Then in step 320, the system may receive bids from the different pharmacies. The system may make a determination to find the closest pharmacy to the user and at the lowest price.

If the pharmacy does not win the prescription, then the system may proceed to step 328 and exit the system. However, there may be a feedback system so that the pharmacy that did not win the bid would receive information as to the amount of the winning bid and may make adjustments to the matrix in response.

In step 322, if the pharmacy does win the bid, then the system may receive the prescription intent 324.

Once the desired pharmacy is chosen or selected by the user, the system proceeds to step 326 to complete the transaction and may proceed to collect payment.

The winning pharmacy may send an invoice or other signal that the user needs to make a payment for the prescription. The user may use the app to make a payment or the user may proceed to complete the transaction and then pay once they arrive at the pharmacy.

In step 326, if the transaction is complete, then the system may send a confirmation for the user in accordance with one or more embodiments of the present invention. If the user cancels the transaction, then the system may proceed to step 328 and exit the system.

If, however, once the transaction is completed, the winning pharmacy would prepare the prescription for the user.

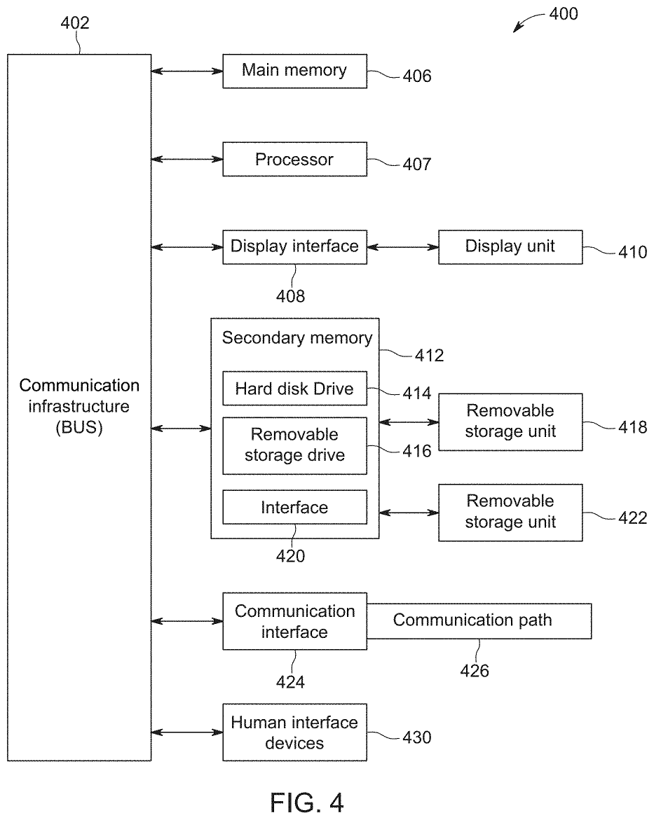

FIG. 4 diagrams a general-purpose computer and peripherals 400, when programmed as described herein, may operate as a specially programmed computer capable of implementing one or more methods, apparatus and/or systems of the solution described in this disclosure. Processor 407 may be coupled to bi-directional communication infrastructure 402 such as communication infrastructure system bus 402. Communication infrastructure 402 may generally be a system bus that provides an interface to the other components in the general-purpose computer system such as processor 407, main memory 406, display interface 408, secondary memory 412 and/or communication interface 424.

Main memory 406 may provide a computer readable medium for accessing and executed stored data and applications. Display interface 408 may communicate with display unit 410 that may be utilized to display outputs to the user of the specially-programmed computer system. Display unit 410 may comprise one or more monitors that may visually depict aspects of the computer program to the user. Main memory 406 and display interface 408 may be coupled to communication infrastructure 402, which may serve as the interface point to secondary memory 412 and communication interface 424. Secondary memory 412 may provide additional memory resources beyond main memory 406, and may generally function as a storage location for computer programs to be executed by processor 407. Either fixed or removable computer-readable media may serve as Secondary memory 412. Secondary memory 412 may comprise, for example, hard disk 414 and removable storage drive 416 that may have an associated removable storage unit 418. There may be multiple sources of secondary memory 412 and systems implementing the solutions described in this disclosure may be configured as needed to support the data storage requirements of the user and the methods described herein. Secondary memory 412 may also comprise interface 420 that serves as an interface point to additional storage such as removable storage unit 422. Numerous types of data storage devices may serve as repositories for data utilized by the specially programmed computer system. For example, magnetic, optical or magnetic-optical storage systems, or any other available mass storage technology that provides a repository for digital information may be used.

Communication interface 424 may be coupled to communication infrastructure 402 and may serve as a conduit for data destined for or received from communication path 426. A network interface card (NIC) is an example of the type of device that once coupled to communication infrastructure 402 may provide a mechanism for transporting data to communication path 426. Computer networks such Local Area Networks (LAN), Wide Area Networks (WAN), Wireless networks, optical networks, distributed networks, the Internet or any combination thereof are some examples of the type of communication paths that may be utilized by the specially program computer system. Communication path 426 may comprise any type of telecommunication network or interconnection fabric that can transport data to and from communication interface 424.

To facilitate user interaction with the specially programmed computer system, one or more human interface devices (HID) 430 may be provided. Some examples of HIDs that enable users to input commands or data to the specially programmed computer may comprise a keyboard, mouse, touch screen devices, microphones or other audio interface devices, motion sensors or the like, as well as any other device able to accept any kind of human input and in turn communicate that input to processor 407 to trigger one or more responses from the specially programmed computer are within the scope of the system disclosed herein.

While FIG. 4 depicts a physical device, the scope of the system may also encompass a virtual device, virtual machine or simulator embodied in one or more computer programs executing on a computer or computer system and acting or providing a computer system environment compatible with the methods and processes of this disclosure. In one or more embodiments, the system may also encompass a cloud computing system or any other system where shared resources, such as hardware, applications, data, or any other resource are made available on demand over the Internet or any other network. In one or more embodiments, the system may also encompass parallel systems, multi-processor systems, multi-core processors, and/or any combination thereof. Where a virtual machine, process, device or otherwise performs substantially similarly to that of a physical computer system, such a virtual platform will also fall within the scope of disclosure provided herein, notwithstanding the description herein of a physical system such as that in FIG. 4.

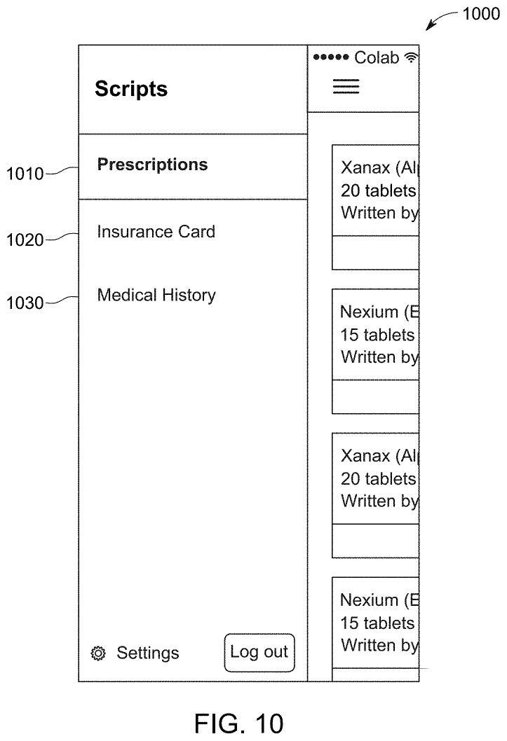

FIG. 10 is a non-limiting example of a graphical user interface 1000 for the user to choose different features in accordance with one or more embodiments of the present invention. In this illustrated example, the user may select to review the status of the user's prescription 1010 or the user may select the insurance card function 1020 to review the user's insurance information. In yet another function, in this illustrated example, the user may select to review the user's medical history 1030.

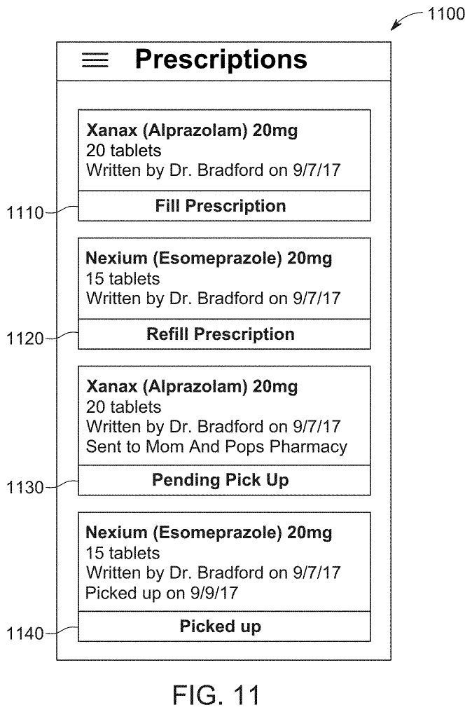

FIG. 11 is a non-limiting example of a graphical user interface 1100 for the user to view the status of the user's prescriptions in accordance with one or more embodiments of the present invention. In this illustrated example, the user may select to fulfill the current prescription 1110 or the user may choose to refill a prescription 1120. As another illustrated example, the user may see that a current prescription is being serviced and may display a pending pick up status 1130. In yet another illustrated example, the prescription page may show that a previous prescription was fulfilled and was picked up 1140.

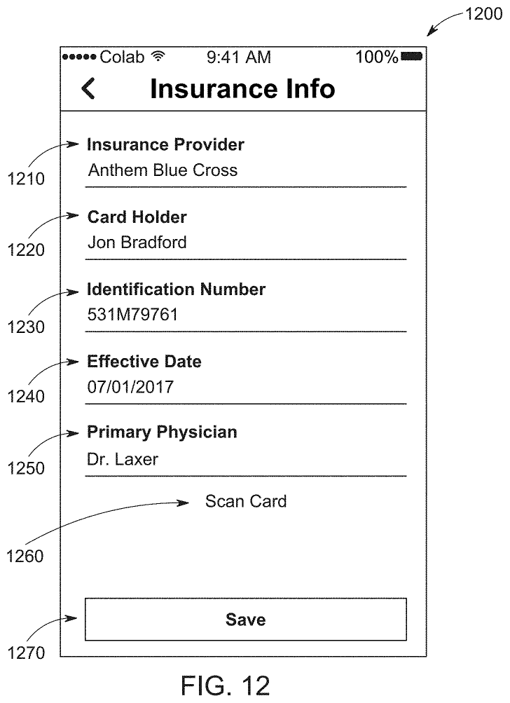

FIG. 12 is a non-limiting example of a graphical user interface 1200 for the user to view the status of the user's insurance information in accordance with one or more embodiments of the present invention. In this illustrated example, the user may see the insurance provider name 1210, card holder's name 1220, insurance identification number 1230, effective date of the policy 1240, or the primary physician's name 1250. Moreover, the insurance information may be collected by selecting the scan card function 1260. If the information entered is accurate, the user may select to save the current information 1270.

FIG. 13 A is a non-limiting example of a graphical user interface 1300 A for the user to view the medical history of the user in accordance with one or more embodiments of the present invention. In this illustrated example, the user may view the first page which may display the user's personal information including full name 1310 A, date of birth 1320 A, phone number 1330 A, address 1340 A. and emergency contact information 1350 A. When the user is done reviewing the personal information they may select to view the next screen 1360 A.

FIG. 13 B is a non-limiting example of a graphical user interface 1300 B for the user to view the medical history of the user in accordance with one or more embodiments of the present invention. In this illustrated example, the user may view the second page which may display the user's current medications 1310 B, and may include information such as medication, dosage, frequency, and the reason for taking the medication. Moreover, the user may see the dates of when certain medical procedures were performed 1320 B, such as a general physical, screening labs, MRI, surgery, mammograms, etc. In addition, the medical history may include condition and symptoms checklist 1330 B and may include information such as skin rash, fatigue, coughing, etc.

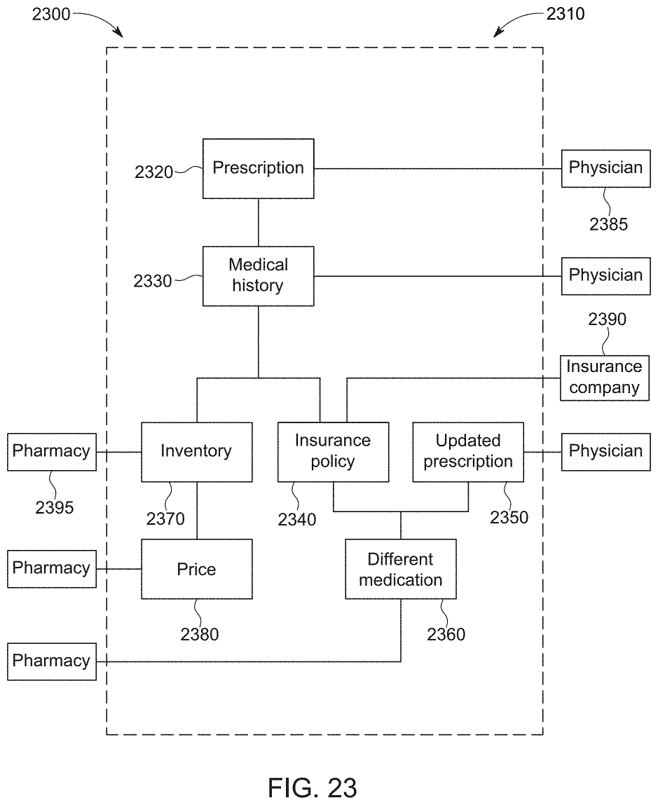

FIG. 23 is a non-limiting example illustrating the concept of the blockchain 2300 aspect of the electronic prescription database as described herein capable of implementing one or more methods, apparatus and/or systems of the present invention.

As a non-limiting example, the electronic prescription database 2310 may be a decentralized storage network. The decentralized storage network may include a digital ledger. As such, different list of records, called blocks may be linked together. Each block is recorded and then added to it in chronological order. Each block may contain a hash pointer as a link to the previous block and a timestamp and transaction date. In the current example, the electronic prescription database may have an updated inventory level 2370 provided by the pharmacy 2395, prescription 2320 written by the physician 2385, updated medical history 2330, user credentials, list of pharmacies proximate to the user, the bid with the lowest price 2380 placed by the pharmacy, the updated price of the prescription, etc. Further, the prescription 2320 itself may be drafted by the physician and entered into the electronic prescription database. Moreover, the changes to the electronic prescription database may be in real-time and the updates may be seen by the different parties as the changes are being made. Each block may be a new prescription 2350, insurance policy 2340 entered by the insurance company 2390, updated inventory level 2370, new medical history 2330, updated or different medication 2360, etc.

In addition, the blocks may be secured by using cryptography. As such, each block may contain a cryptographic pointer as a link to a previous block. Using cryptography, the network may provide a more secure communication system and prevent unauthorized use.

The decentralized network may have different nodes. The node may be a computer connected to the network and may receive a copy of the blockchain. Because there are vast networks of nodes, this may prevent cracks in the system. Since the network is decentralized, it may be difficult to corrupt or hack the system. In order to alter the records, all of the subsequent blocks and the collusion of the network would have to be changed. As such, it may be difficult to corrupt the digital ledger. The decentralized storage network may use ad-hoc message passing and distributed networking.

Ad-hoc message passing may include a message that is sent to a process and relies on the process and the supporting infrastructure to select and invoke the actual code to run. Message passing may be used as a way for the object that make up a program to work with each other and as a means for objects and systems running on different computers, such as the internet, to interact. Message passing may be implemented by various mechanisms, including channels.

Message passing is a technique for invoking behavior (i.e., running a program) on a computer. In contrast to the traditional technique of calling a program by name, message passing uses an object model to distinguish the general function from the specific implementations. The invoking program sends a message and relies on the object to select and execute the appropriate code. The justifications for using an intermediate layer essentially falls into two categories: encapsulation and distribution.

Encapsulation may include bundling of data with the methods that operate on that data. Encapsulation is used to hide the values or state of a structured data object inside a class, preventing unauthorized parties' direct access to them. Publicly accessible methods are generally provided in the class to access the values, and other client classes call these methods to retrieve and modify the values within the object.

Distributed networking may include a distributed computing network system, said to be distributed when the computer programming and the data to be worked on are spread out across more than one computer. Usually, this is implemented over a computer network.

Moreover, the electronic prescription database may be private or semi-private. A private electronic prescription database may require an invitation and validation by either the network starter or by a set of rules placed by the network starter. Moreover, the network may be a permissioned network, which places restrictions on who is allowed to participate in the network and under certain types of entries. As a non-limiting example, the participant may require an invitation or permission to join. Having such a safeguard may be advantageous because it may limit the database to certain types of entities such as physicians, pharmacies, insurance companies, etc.

The electronic prescription database may also be semi-private and may be run by a single company or entity that may grant access to certain types of users. As such, a system may be implemented wherein a set of prequalified entities may gain access to the electronic prescription database. As a non-limiting example, the electronic prescription database may only be accessible for certain physicians in a particular insurance network. Then again, there may be certain types of pharmacies that are part of a larger company or network that may have access to a certain electronic prescription database.

The electronic prescription database may be Health Insurance Portability and Accountability Act (HIPAA) compliant. As such, the electronic prescription database may put into place procedures and policies for maintaining the privacy and the security of individual identifiable health information. More specifically, there are three types of security safeguards required for compliance, administrative, physical, and technical. The administrative safeguards concern the policies and procedures designed to show how the entity will comply with HIPAA.

The physical safeguards concern controlling physical access to protect against inappropriate access to protected data.

The technical safeguards concern controlling access to computer systems and enabling covered entities to protect communications containing Protected Health Information (PHI) transmitted electronically over open networks from being intercepted by anyone other than the intended recipient.

This may provide for the prevention of fraud and abuse within the electronic prescription database.

While the invention herein disclosed has been described by means of specific embodiments and applications thereof, numerous modifications and variations could be made thereto by those skilled in the art without departing from the scope of the invention set forth in the claims.

What is claimed is:

1. A method for a portable electronic prescription comprising:
receiving login credentials of a user for an electronic prescription database, the electronic prescription database is a decentralized storage network, wherein the decentralized storage network comprises a digital ledger;
validating the user's credentials with the electronic prescription database;
receiving a prescription for the user;
using the prescription in a search of the electronic prescription database to obtain a list of pharmacies with the lowest prices for the prescription and the location of the listed pharmacies;
sending the listed pharmacies, their prices and locations for display to the user;
receiving a pharmacy intent based on a selection by the user; and
completing a transaction between the user and the selected pharmacy.

2. The method of claim 1, wherein the prescription comprises an electronically converted handwritten prescription.

3. The method of claim 1, wherein the prescription comprises the user's medical records.

4. The method of claim 1, wherein the user's prescription is refillable.

5. The method of claim 1, wherein the electronic prescription database comprises a real-time inventory of the prescription.

6. The method of claim 1, wherein the listed pharmacies further comprises the user's last used pharmacy.

7. The method of claim 1, wherein the location of the listed pharmacy is determinable by global positioning satellite.

8. The method of claim 1, wherein the location of the listed pharmacy is determinable by geofencing.

9. The method of claim 1, wherein the login credentials comprises the user's Username and Password for the electronic prescription database.

10. The method of claim 1, wherein the prescription comprises one or more of a first name, last name, date of birth, social security number, driver's license number, domicile, medication prescription, and prescription history.

11. A method for a portable electronic prescription comprising:
receiving login credentials of a user for an electronic prescription database, the electronic prescription database is a decentralized storage network, wherein the decentralized storage network comprises a digital ledger;
validating the user's credentials with the electronic prescription database;
receiving a prescription for the user;
locating a pharmacy proximate to a location of the user;
using the prescription in a search of the electronic prescription database to obtain a list of pharmacies with the lowest prices for the prescription, real-time internal inventory, the location of the listed pharmacies, and driving directions to the listed pharmacies;
sending the listed pharmacies, their prices, real-time internal inventories, locations, driving directions for display to the user;
receiving the user's pharmacy intent based on a selection by the user; and
completing the transaction between the user and the selected pharmacy.

12. The method of claim 11, wherein the portable electronic prescription comprises an electronically converted handwritten prescription.

13. The method of claim 11, wherein the prescription comprises of the user's medical records.

14. The method of claim 11, wherein the user's portable electronic prescription is refillable.

15. The method of claim 11, wherein the listed pharmacies further comprises the user's last used pharmacy.

16. The method of claim 11, wherein the location of the listed pharmacy is determinable by global positioning satellite.

17. The method of claim 11, wherein the location of the listed pharmacy is determinable by geofencing.

18. The method of claim 11, wherein the login credentials comprises the user's Username and Password for the electronic prescription database.

19. The method of claim 11, wherein the prescription comprises one or more of a first name, last name, date of birth, social security number, driver's license number, domicile, medication prescription, and prescription history.

20. A method for bidding for a prescription comprising:
receiving login credentials of a pharmacy for an electronic prescription database, the electronic prescription database is a decentralized storage network, wherein the decentralized storage network comprises a digital ledger;
validating the pharmacy's credentials with the electronic prescription database;
sending the prescription for a user,
using a location of the user to locate a proximate pharmacy;
checking a real-time internal inventory of the pharmacy proximate to the location of the user;
bidding with a price for the prescription based on a predetermined pricing matrix with one or more drug manufacturers;
receiving a bid with the price of the prescription and real-time internal inventory level of the prescription for the pharmacy proximate to the location of the user for display to the user;
sending a user's pharmacy intent based on a selection by the user; and
completing the transaction between the user and the selected pharmacy.
